(19) United States
(12) Patent Application Publication
(10) Pub. No.: US 2025/0034095 A1
(43) Pub. Date: Jan. 30, 2025

(54) NEW PROCESS FOR PREPARATION OF AFICAMTEN

(71) Applicant: Zydus Lifesciences Ltd., Gandhinagar (IN)

(72) Inventors: Kumar Kamlesh Singh, Gandhinagar (IN); Jitesh Amratlal Desai, Gandhinagar (IN); Chirag Jayantilal Vyas, Gandhinagar (IN); Jaydev Rameshchandra Vaghela, Gandhinagar (IN)

(21) Appl. No.: 18/767,551

(22) Filed: Jul. 9, 2024

(30) Foreign Application Priority Data
Jul. 10, 2023 (IN) 202321046243

(51) Int. Cl. C07D 231/12

(52) U.S. Cl. CPC C07D 231/12; C07B 2200/09

(57) ABSTRACT

The present disclosure provides a new process for preparation of Aficamten. The present disclosure also provides t a novel intermediate compound or a salt, isomer or mixture thereof. Further, the present disclosure also provides a process for preparation of novel intermediate compound or a salt, an isomer or a mixture thereof. Furthermore, the provides a method of using of novel intermediate compound for preparation of Aficamten.

### CROSS REFERENCE TO RELATED APPLICATION

This application claims priority under § 119(a) from Indian Patent Application No. 202321046243, filed Jul. 10, 2023, which is incorporated by reference herein in its entirety.

### FIELD OF THE DISCLOSURE

The present disclosure provides a new process for preparation of Aficamten. The present disclosure also provides a novel intermediate compound or a salt, an isomer or a mixture thereof. Further, the present invention provides a process for preparation of novel intermediate compound or a salt, an isomer or a mixture thereof. Furthermore, the present disclosure provides use of novel intermediate compound for preparation of Aficamten.

### BACKGROUND

The following discussion of the related art is intended to present the invention in an appropriate technical context and allow its significance to be properly appreciated. Unless clearly indicated to the contrary, reference to any prior art in this specification should be construed as such art is widely known or forms part of common general knowledge in the field.

Aficamten is an investigational, oral, small molecule cardiac myosin inhibitor and is chemically known as N-[(1R)-5-(5-ethyl-1,2,4-oxadiazol-3-yl)-2,3-dihydro-1H-inden-1-yl]-1-methylpyrazole-4-carboxamide, having chemical structure as shown below:

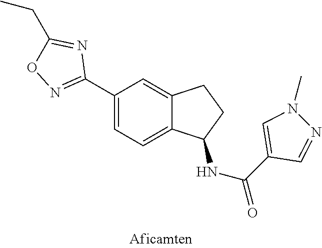 [SMILES: CCc1nc(-c2ccc3c(c2)CC[C@H]3NC(=O)c2cnn(C)c2)no1]

International (PCT) Publication No. WO2019/144041 discloses process for preparation of Aficamten and intermediate compounds thereof.

International (PCT) Publication No. WO2021/011807 discloses process for preparation of Aficamten and polymorphic forms thereof.

International (PCT) Publication No. WO2023/015184 discloses process for preparation of Aficamten and intermediates thereof.

As of now, there are very limited disclosures are available for synthesis of Aficamten and intermediates thereof. Therefore, there is a need to develop cost effective novel processes for preparation of Aficamten containing novel intermediate compounds, which results into high overall yield and purity.

The present invention relates new process for preparation of Aficamten. Further, the present invention relates to novel intermediate compounds or a salt, an isomer or a mixture thereof and their process for preparation. Furthermore, the present invention relates to use of novel intermediate compounds or a salt, an isomer or a mixture thereof for preparation of Aficamten.

### SUMMARY

The process according to the present invention can generally be depicted according to below Scheme I:

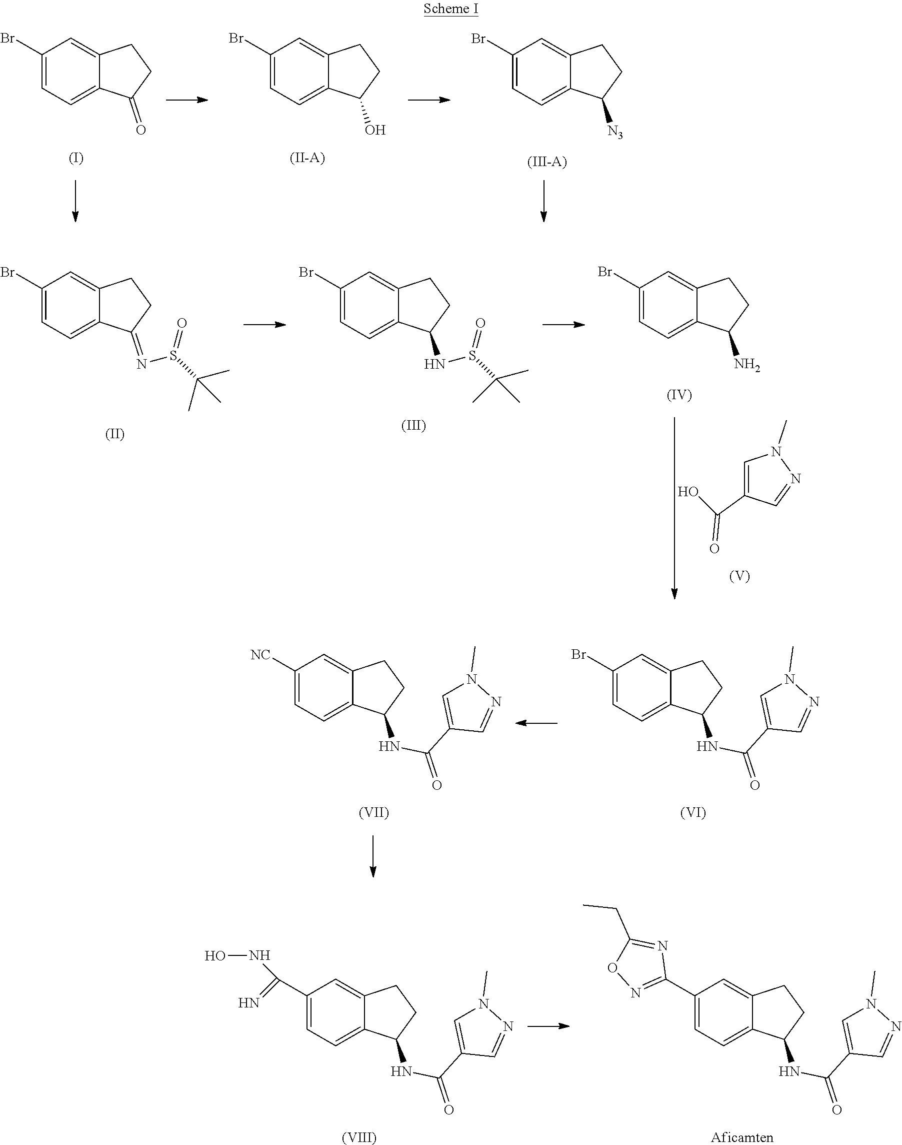

In one general aspect, there is provided a novel compound of Formula (VI) or a salt, isomer, or mixture thereof.

In another general aspect, there is provided a use of compound of Formula (VI) or a salt, an isomer or a mixture thereof, for preparation of Aficamten.

In another general aspect, there is provided a process for preparation of a compound Formula (VI) or a salt, an isomer or a mixture thereof, comprising: converting a compound of Formula (IV) or a salt, an isomer or a mixture thereof to a compound of Formula (VI) or a salt, an isomer or a mixture thereof.

In another general aspect, there is provided a process for preparation of a compound Formula (VI) or a salt, an isomer or a mixture thereof comprising: reacting a compound of Formula (IV) or a salt, an isomer or a mixture thereof with a compound of Formula (V) to obtain compound Formula (VI) or a salt, an isomer or a mixture thereof.

In another general aspect, there is provided a process for preparation of a compound Formula (VI) or a salt, an isomer or a mixture thereof, comprising:
(a) converting a compound of Formula (I) to a compound of Formula (II-A) or a salt, an isomer or a mixture thereof,
(b) converting the compound of Formula (II-A) or a salt, an isomer or a mixture thereof to a compound of Formula (III-A) or a salt, an isomer or a mixture thereof,
(c) converting the compound of Formula (III-A) or a salt, an isomer or a mixture thereof to a compound of Formula (IV) or a salt, an isomer or a mixture thereof, and
(d) reacting the compound of Formula (IV) to or a salt, an isomer or a mixture thereof with a compound of Formula (V) to obtain the compound of Formula (VI) or a salt, an isomer or a mixture thereof.

In another general aspect, there is provided a process for preparation of a compound Formula (VI) or a salt, an isomer or a mixture thereof, comprising:
(a) converting a compound of Formula (I) to a compound of Formula (II) or a salt, an isomer or a mixture thereof,
(b) reducing the compound of Formula (II) or a salt, an isomer or a mixture thereof to a compound of Formula (III) or a salt, an isomer or a mixture thereof,
(c) converting the compound of Formula (III) or a salt, an isomer or a mixture thereof to a compound of Formula (IV) or a salt, an isomer or a mixture thereof, and
(d) reacting the compound of Formula (IV) to or a salt, an isomer or a mixture thereof with compound of Formula (V) to obtain the compound of Formula (VI) or a salt, an isomer or a mixture thereof.

In another general aspect, there is provided a process for preparation of compound of Formula (VII) comprising, converting a compound of Formula (VI) or a salt, an isomer or a mixture thereof to the compound of Formula (VII) or a salt, an isomer or a mixture thereof.

In another general aspect, there is provided a process for preparation of a compound of Formula (VII) comprising:
(a) converting a compound of Formula (IV) or a salt, an isomer or a mixture thereof to a compound of Formula (VI) or a salt, an isomer or a mixture thereof, and
(b) converting the compound of Formula (VI) or a salt, an isomer or a mixture thereof to the compound of Formula (VII) or a salt, an isomer or a mixture thereof.

In another general aspect, there is provided a use of a compound of Formula (VI) or a salt, an isomer or a mixture thereof for preparation of Aficamten.

In another general aspect there is provided a process for preparation of Aficamten comprising:
(a) converting a compound of Formula (VI) or a salt, an isomer or a mixture thereof to a compound of Formula (VII) or a salt, an isomer or a mixture thereof,
(b) converting the compound of Formula (VII) or a salt, an isomer or a mixture thereof to a compound of Formula (VIII) or a salt, an isomer or a mixture thereof, and
(c) converting the compound of Formula (VIII) or a salt, an isomer or a mixture thereof to Aficamten.

In another general aspect there is provided a process for preparation of Aficamten comprising:
(a) reacting a compound of Formula (IV) or a salt, an isomer or a mixture thereof with a compound of Formula (V) to obtain a compound of Formula (VI) or a salt, an isomer or a mixture thereof,
(b) converting the compound of Formula (VI) or a salt, an isomer or a mixture thereof to a compound of Formula (VII) or a salt, an isomer or a mixture thereof,
(c) converting the compound of Formula (VII) or a salt, an isomer or a mixture thereof to a compound of Formula (VIII) or a salt, an isomer or a mixture thereof, and
(d) converting the compound of Formula (VIII) or a salt, an isomer or a mixture thereof to Aficamten.

In another general aspect there is provided a process for preparation of Aficamten comprising:
(a) converting a compound of Formula (I) to a compound of Formula (IV) or a salt, an isomer or a mixture thereof,
(b) reacting the compound of Formula (IV) or a salt, an isomer or a mixture thereof with a compound of Formula (V) to obtain a compound of Formula (VI) or a salt, an isomer or a mixture thereof,
(c) converting the compound of Formula (VI) or a salt, an isomer or a mixture thereof to a compound of Formula (VII) or a salt, an isomer or a mixture thereof,
(d) converting the compound of Formula (VII) or a salt, an isomer or a mixture thereof to a compound of Formula (VIII) or a salt, an isomer or a mixture thereof, and
(e) converting the compound of Formula (VIII) or a salt, an isomer or a mixture thereof to Aficamten.

In another general aspect there is provided a process for preparation of Aficamten comprising:
(a) converting a compound of Formula (I) to a compound of Formula (III) or a salt, an isomer or a mixture thereof,
(b) converting the compound of Formula (III) or a salt, an isomer or a mixture thereof to a compound of Formula (IV) or a salt, an isomer or a mixture thereof,
(c) reacting the compound of Formula (IV) or a salt, an isomer or a mixture thereof with a compound of Formula (V) to obtain a compound of Formula (VI) or a salt, an isomer or a mixture thereof,
(d) converting the compound of Formula (VI) or a salt, an isomer or a mixture thereof to a compound of Formula (VII) or a salt, an isomer or a mixture thereof,
(e) converting the compound of Formula (VII) or a salt, an isomer or a mixture thereof to a compound of Formula (VIII) or a salt, an isomer or a mixture thereof, and
(f) converting the compound of Formula (VIII) or a salt, an isomer or a mixture thereof to Aficamten.

In another general aspect there is provided a process for preparation of Aficamten comprising:
(a) converting a compound of Formula (I) to a compound of Formula (II-A) or a salt, an isomer or a mixture thereof,
(b) converting the compound of Formula (II-A) to a compound of Formula (III-A) or a salt, an isomer or a mixture thereof
(c) converting the compound of Formula (III-A) or a salt, an isomer or a mixture thereof to a compound of Formula (IV) or a salt, an isomer or a mixture thereof,
(d) reacting the compound of Formula (IV) or a salt, an isomer or a mixture thereof with a compound of Formula (V) to obtain a compound of Formula (VI) or a salt, an isomer or a mixture thereof,
(e) converting the compound of Formula (VI) or a salt, an isomer or a mixture thereof to a compound of Formula (VII) or a salt, an isomer or a mixture thereof,
(f) converting the compound of Formula (VII) or a salt, an isomer or a mixture thereof to a compound of Formula (VIII) or a salt, an isomer or a mixture thereof, and
(g) converting the compound of Formula (VIII) or a salt, an isomer or a mixture thereof to Aficamten.

In another general aspect, the process of present invention provides Aficamten having a purity of about 98% or more by area percentage of HPLC. In particular, Aficamten having a purity of about 99% or more, more particularly, a purity of about 99.5% or more, further more particularly, a purity of about 99.8% or more, most particularly, a purity of about 99.9% or more, by area percentage of HPLC.

In another general aspect, the process of present invention provides Aficamten having a chiral purity of about 98% or more, by area percentage of HPLC. In particular, aficamten having a chiral purity of about 99% or more, more particularly, a chiral purity of about 99.5% or more, furthermore particularly, a chiral purity of about 99.8% or more, most particularly, a chiral purity of about 99.9% or more, by area percentage of HPLC.

In another general aspect, the present invention provides a composition comprising Aficamten and pharmaceutically acceptable carrier.

In another general aspect, there is provided a composition comprising Aficamten having a purity of about 99% or more as determined by high performance liquid chromatography (HPLC).

In another general aspect, there is provided a composition comprising Aficamten having a chiral purity of about 99% or more as determined by high performance liquid chromatography (HPLC).

### Definitions

The term “solution” as used herein, unless described otherwise, does not necessarily mean only a clear solution or one wherein the solute is completely soluble in the solvent; all the intermediate phases of mixture of components, starting from a state wherein the solute has stated getting dissolved in the solvent to a state wherein the solute has completely dissolved in the solvent, are included within the expression “solution.”

As used herein, the term “area percentage of HPLC” refers to the area percentage of a peak in HPLC chromatogram. In some embodiments, an area percentage is relative to the total area of the composition in a HPLC chromatogram. In some embodiments, an area percentage is relative to the area of Aficamten in a HPLC chromatogram.

The terms “obtaining,” “isolating,” and “purifying” are generally interchangeable and include, but not limited to, decantation, filtration, extraction, evaporation, crystallization, recrystallization and chromatographic operations.

Optionally, the solution, prior to any solids formation and/or solvent removal, can be filtered to remove any undissolved solids, particulate matter or solid impurities. Any filtration system and filtration techniques known in the art can be used for the purpose.

Terms such as “about” and “generally” are to be construed as modifying a term or value to which they are attached such that the term or the value is not absolute. This includes, at very least, the degree of expected experimental error, technique error and instrument error for a given technique used to measure a value.

The term “pharmaceutically acceptable” as used herein is intended to mean that it is useful in preparing a pharmaceutical composition that is generally non-toxic, is not biologically undesirable, and includes that which is acceptable for veterinary use and/or human pharmaceutical use.

The term “salt” or “salts” includes salts with an inorganic acid e.g. hydrochloric acid, hydroiodic acid, phosphoric acid, phosphonic acid, sulfuric acid, hydrobromic acid or with an organic acid e.g. formic acid, acetic acid, citric acid, malic acid, maleic acid, tartaric acid, succinic acid, hemisuccinic acid, salicylic acid, trifluoroacetic acid, trichloroacetic acid, oxalic acid, benzoic acid, p-toluene sulfonic acid or methanesulfonic acid.

The term “composition” used herein means a physical mixture of two or more components.

The term “pharmaceutical composition” is intended to encompass a drug product including the active ingredient(s), pharmaceutically acceptable excipients that make up the carrier, as well as any product, which results, directly or indirectly, from combination, complexation or aggregation of any two or more of the ingredients.

Accordingly, the pharmaceutical compositions encompass any composition made by admixing the active ingredient, active ingredient dispersion or composite, additional active ingredient(s), and pharmaceutically acceptable excipients.

As used herein, the phrase “not in detectable amount,” refers to the level of impurity in the product, which is below the level of detection limit, when analyzed using the HPLC method.

The terms used throughout the description is defined herein below.
“TEA” refers to Triethyl amine.
“MTBE” refers to methyl tert-butyl ether.
“NaOH” refers to sodium hydroxide.
“KOH” refers to potassium hydroxide.
“NaBH₄” refers to sodium borohydride.
“NaHCO₃” refers to sodium bicarbonate.
“Na₂CO₃” refers to sodium carbonate.
“K₂CO₃” refers to potassium carbonate.
“K₃PO₄” refers to tripotassium phosphate.
“DMF” refers to N,N-dimethylformamide.
“NMP” refers to N-methyl pyrrolidone.
“NMM” refers to N-methylpiperidine.
“HOBt” refers to 1-hydroxybenzotriazole.
“HOAt” refers to 1-hydroxy-7-azabenzotriazole.
“HATU” refers to (N-[(dimethylamino)-1H-1,2,3-triazolo[4,5-b]pyridin-1-ylmethylene]-N-methylmethanaminium hexafluorophosphate N-oxide.
“HBTU” refers to N-[(1H-Benzotriazol-1-yl)(dimethylamino)methylene]-N-methyl methanaminium hexafluorophosphate N-oxide.
“TBTU” refers to (N-[(1Hbenzotriazol-1-yl)(dimethylamino)methylene]-N-methyl methanaminium tetrafluoroborate N-oxide.
“TPTU” refers to (2-(2-oxo-1(2H)-pyridyl-1,1,3,3-tetramethyluronium tetrafluoroborate).
“TOTU” refers to (O-[(cyano(ethoxycarbonyl)methyleneamino]-N,N,N′,N′-tetramethyl uronium tetrafluoroborate).
“BOP” refers to ((benzotriazol-1-yloxy)tris(dimethylamino)phosphonium hexafluoro phosphate.
“CDMT” refers to 2-chloro-4,6-dimethoxy-1,3,5-triazine.
“DMTMM” refers to 4-(4,6-dimethoxy-1,3,5-triazin-2-yl)-4-methylmorpholinium chloride.
“EDC” refers to 1-Ethyl-3-(3-dimethylaminopropyl)carbodiimide.
“EDC·HCl” refers to 1-Ethyl-3-(3-dimethylaminopropyl)carbodiimide hydrochloride.
“SOCl₂” refers to thionyl chloride.
“DCC” refers to N,N′-dicyclohexane carbodiimide.
“DIC” refers to N,N′-diisopropylcarbodiimide.
“Boc” refers to di-tert-butyl decarbonate.
“T3P” refers to propanephosphonic acid anhydride.
“MTBE” refers to methyl tert-butyl ether.
“EMPA” refers to ethylmethylphosphinic anhydride.
“EEDQ” refers to 2-ethoxy-1-ethoxycarbonyl-1,2-dihydroquinoline.
“CDI” refers to carbonyldiimidazole.
“THF” refers to tetrahydrofuran.
“DMF” refers to dimethylformamide.
“DME” refers to 1,2-dimethoxyethane
“HPLC” refers to High Performance Liquid Chromatography.

### DETAILED DESCRIPTION

The aforementioned general and further aspects of the invention are achieved by the detailed description of the invention provided herein after.

While the invention has been described in terms of its specific aspects, the description, in no way, intends to limit the scope of the present invention to the specifically described aspects only; equivalents and variants thereof which are apparently obvious to those skilled in the art are also included within the scope of the present invention.

The description does not include detailed description of conventional methods used in the field of the invention; such conventional methods are the ones well known to those of ordinary skill in the art either because they are normally practiced routinely by the skilled artisan and/or are described in detail in various publications-physical as well digital.

The product(s) obtained may further be purified to obtain them in purer form.

The product(s) obtained may further be dried additionally to achieve desired level of moisture and/or residual solvents.

The product(s) obtained may further be converted to any other physical forms thereof which includes but not specifically limited to polymorph(s), salt(s), solvate(s), hydrate(s), co-crystal(s) or solid dispersion(s); and solid forms thereof.

The product(s) obtained may further be subjected to physical processing which includes, but not limited to, pressing, crushing, triturating, milling or grinding to adjust the particle size of the product(s) to desired levels.

The product(s) obtained may further be combined with pharmaceutically acceptable career to obtain a pharmaceutical composition comprising one or more of the solid-state forms of Aficamten of the invention and pharmaceutically acceptable carriers, excipients or diluents. The pharmaceutical composition may be in the form of solid or liquid dosage forms and may have immediate release or modified release characteristics. The dosage forms include but not limited to tablet, capsule, powder, granules, solution, suspension, emulsion, elixir or cream.

In one general aspect, there is provided a compound of Formula (VI), or a salt, an isomer, or a mixture thereof,

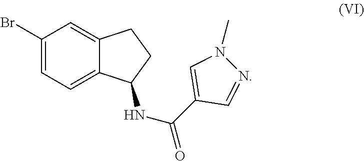 [SMILES: Cn1cc(C(=O)N[C@@H]2CCc3cc(Br)ccc32)cn1]

In another general aspect, there is provided use of a compound of Formula (VI) or a salt, an isomer, or a mixture thereof, for the preparation of Aficamten.

In another general aspect, there is provided a process for the preparation of a compound of Formula (VI) or a salt, an isomer, or a mixture thereof,

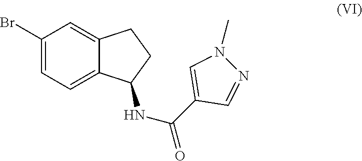 [SMILES: Cn1cc(C(=O)N[C@@H]2CCc3cc(Br)ccc32)cn1]

the process comprising reacting a compound of Formula (IV) or a salt, an isomer, or a mixture thereof,

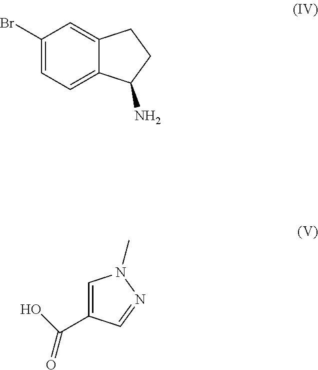

with a compound of Formula (V) to obtain the compound Formula (VI) or a salt, an isomer, or a mixture thereof.

In general, the condensation of the compound of Formula (IV) or a salt, an isomer or a mixture thereof with the compound of Formula (V) may be performed in the presence of a coupling agent, a base and solvent or a mixture of solvents, wherein the coupling agent is selected from HOBt, HOAt, HATU, HBTU, TBTU, TPTU, TOTU, EDC, EDC·HCl, DCC, DIC, CDI, SOCl₂, oxalyl chloride, Vilsmeier reagent, phosphorous oxychloride, or a mixture thereof. In particular, the coupling agent is EDC·HCl. The base is selected from imidazole, TEA, DIPEA, NMM, pyridine, NaOH, NaHCO₃, Na₂CO₃, K₂CO₃, K₃PO₄, N-methylpiperidine, or a mixture thereof. In particular, the base is DIPEA. The solvent is selected from DMF, dimethylacetamide, ethyl acetate, ethanol, methanol, propanol, isopropyl alcohol, NMP, dichloromethane, toluene, THF, n-heptane, n-hexane, dioxane, di-n-butyl ether, acetonitrile, MTBE, DME, water, or a mixture thereof. In particular, the solvent is DMF.

In another general aspect, there is provided a process for the preparation of a compound of Formula (VI) or a salt, an isomer or a mixture thereof, the process comprising:

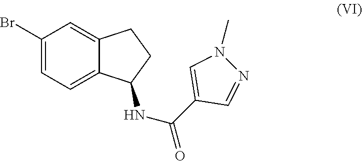 [SMILES: Cn1cc(C(=O)N[C@@H]2CCc3cc(Br)ccc32)cn1]

(a) converting a compound of Formula (I) to a compound of Formula (II-A) or a salt, an isomer or a mixture thereof,

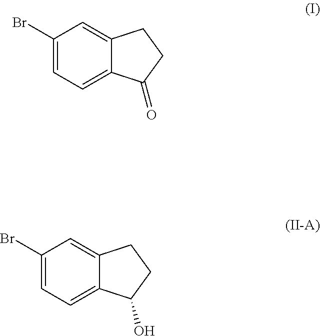 [SMILES: O=C1CCc2cc(Br)ccc21 ; O[C@H]1CCc2cc(Br)ccc21]

(b) converting the compound of Formula (II-A) or a salt, an isomer or a mixture thereof to a compound of Formula (III-A) or a salt, an isomer or a mixture thereof,

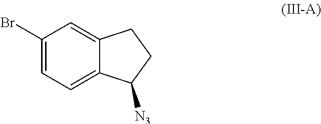 [SMILES: [N-]=[N+]=N[C@@H]1CCc2cc(Br)ccc21]

(c) converting the compound of Formula (III-A) or a salt, an isomer or a mixture thereof to a compound of Formula (IV) or a salt, an isomer or a mixture thereof, and

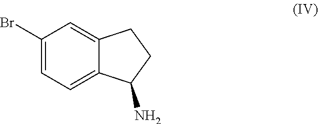 [SMILES: N[C@@H]1CCc2cc(Br)ccc21]

(d) reacting the compound of Formula (IV) to or a salt, an isomer or a mixture thereof with a compound of Formula (V) to obtain the compound of Formula (VI) or a salt, an isomer or a mixture thereof.

Optionally, the process of this aspect may be performed in situ without isolation of the compound of Formula (II-A) and/or compound of Formula (III-A).

In general, the reduction at step (a) may be performed by using a reducing agent in the presence of a catalyst and a solvent or a mixture of solvents, wherein the reducing agent is selected from borane dimethylsulfide, borane-tetrahydrofuran, borane-N,N-diethylaniline, diborane, or a mixture thereof. In particular, the reducing agent is borane dimethylsulfide. The catalyst is (R)-2-methyl-CBS-oxazaborolidine. The solvent is selected from DMF, dimethylacetamide, ethyl acetate, ethanol, methanol, propanol, isopropyl alcohol, NMP, dichloromethane, toluene, THF, n-heptane, n-hexane, dioxane, di-n-butyl ether, acetonitrile, MTBE, DME, water, chloroform, or a mixture thereof. In particular, the solvent is dichloromethane.

In general, the reduction at step (b) may be performed by using a reagent in the presence of a catalyst and a solvent or a mixture of solvents, wherein the reagent is selected from diphenyl phosphoryl azide (DPPA), diethyl phosphoryl azide, di(n-butyl) phosphoryl azide, di(p-methoxyphenyl) phosphoryl azide, triphenylphosphine or a mixture thereof. In particular, the reagent is DPPA. The catalyst is selected from 1,8-diazabicyclo[5.4.0] undec-7-ene (DBU), 1,4-diazabicyclo[2.2.0]octane (DABCO), 1,5-diazabicyclo[4.3.0] non-5-ene (DBN) or a mixture thereof. In particular, the catalyst is DBU. The solvent is selected from dichloromethane, THF, toluene, benzene, hexane, MDC, chloroform, diethyl ether, dioxane, tetrahydrofuran, DMF, DEF, acetonitrile, water, methanol, ethanol, isopropanol, n-butanol, acetone, methyl ethyl ketone, methyl isobutyl ketone, ethyl acetate, isopropyl acetate, butyl acetate, or a mixture thereof. In particular, the solvent is toluene.

In general, the reduction of azide at step (c) may be performed by using a reagent in presence of a solvent or mixture of solvents, wherein the reagent is selected from triphenylphosphine, tributyphosphine, Pd/C, zinc chloride, stannous chloride, ammonium chloride, or a mixture thereof. In particular, the reagent is stannous chloride dihydrate. The solvent is selected from water, methanol, ethanol, propanol, isopropanol, ethyl acetate, THF or a mixture thereof. In particular, the solvent is methanol. Optionally, the reduction at step (c) may be performed in the presence of a catalyst.

In general, the condensation reaction at step (d) may be performed by using a coupling agent in the presence of a base and a solvent or a mixture of solvents, wherein the coupling agent is selected from HOBt, HOAt, HATU, HBTU, TBTU, TPTU, TOTU, EDC, EDC·HCl, DCC, DIC, CDI, SOCl₂, oxalyl chloride, Vilsmeier reagent, phosphorous oxychloride, or a mixture thereof. In particular, the coupling agent is EDC·HCl with HOBt. The base is selected from imidazole, TEA, DIPEA, NMM, pyridine, NaOH, NaHCO₃, Na₂CO₃, K₂CO₃, K₃PO₄, N-methylpiperidine or a mixture thereof. In particular, the base is DIPEA. The solvent is selected from DMF, dimethylacetamide, ethyl acetate, ethanol, methanol, propanol, isopropyl alcohol, NMP, dichloromethane, toluene, THF, n-heptane, n-hexane, dioxane, di-n-butyl ether, acetonitrile, MTBE, DME, water, or a mixture thereof. In particular, the solvent is DMF.

In another general aspect, there is provided a process for preparation of compound of Formula (VI) or a salt, an isomer or a mixture thereof, comprising:

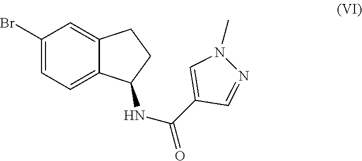 [SMILES: Cn1cc(C(=O)N[C@@H]2CCc3cc(Br)ccc32)cn1]

(a) converting a compound of Formula (I) to compound of Formula (II) or a salt, an isomer or a mixture thereof,

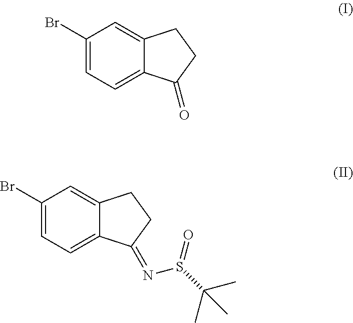 [SMILES: O=C1CCc2cc(Br)ccc21 ; CC(C)(C)[S@@](=O)/N=C1\CCc2cc(Br)ccc21]

(b) reducing the compound of Formula (II) or a salt, an isomer or a mixture thereof to a compound of Formula (III) or a salt, an isomer or a mixture thereof,

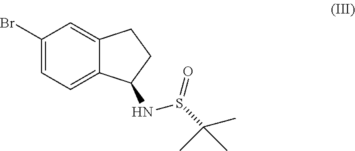 [SMILES: CC(C)(C)[S@@](=O)N[C@@H]1CCc2cc(Br)ccc21]

(c) converting the compound of Formula (III) or a salt, an isomer or a mixture thereof to a compound of Formula (IV) or a salt, an isomer or a mixture thereof, and

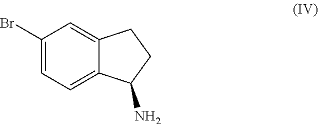 [SMILES: N[C@@H]1CCc2cc(Br)ccc21]

(d) reacting the compound of Formula (IV) to or a salt, an isomer or a mixture thereof with a compound of Formula (V),

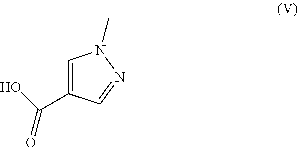

to obtain the compound of Formula (VI) or a salt, an isomer or a mixture thereof.

Optionally, the process of this aspect may be performed in situ without isolation of the compound of Formula (II) and/or compound of Formula (III).

The reaction at step (a) involve condensation of the compound of Formula (I) with tert-butane sulfinamide or isomer thereof to obtain the compound of Formula (II) or a salt, an isomer or a mixture thereof.

In general, the condensation at step (a) may be performed in presence of a Lewis acid and a solvent or a mixture of solvents, wherein the Lewis acid is selected from titanium tetraethoxide, titanium (IV) isopropoxide, aluminium (III) isopropoxide, zirconium (IV) isopropoxide, titanium (IV) methoxide, or mixture thereof. In particular, the Lewis acid is titanium tetraethoxide. The solvent is selected from water, methanol, ethanol, isopropanol, n-butanol, acetone, methyl ethyl ketone, methyl isobutyl ketone, acetonitrile, dimethyl formamide, N,N-dimethyl acetamide, dimethyl sulfoxide, N-methyl pyrrolidone, ethyl acetate, isopropyl acetate, butyl acetate, toluene, xylene, ethyl benzene, methylene dichloride, chlorobenzene, tetrahydrofuran, 2-methyl tetrahydrofuran, methyl tert-butyl ether, di-tert-butyl ether, diisopropyl ether, diethyl ether, cyclohexane, 1,4-dioxane or mixture thereof. In particular, the solvent is toluene.

In general, the reduction reaction at step (b) may be performed in presence of a reducing agent and a solvent or a mixture of solvents, wherein the reducing agent is selected from sodium borohydride, lithium borohydride, sodium hydride, lithium hydride, or a mixture thereof. In particular, the reducing agent is sodium borohydride. The solvent is selected from water, methanol, ethanol, isopropanol, n-butanol, acetone, methyl ethyl ketone, methyl isobutyl ketone, acetonitrile, dimethyl formamide, N,N-dimethyl acetamide, dimethyl sulfoxide, N-methyl pyrrolidone, ethyl acetate, isopropyl acetate, butyl acetate, toluene, xylene, ethyl benzene, methylene dichloride, chlorobenzene, tetrahydrofuran, 2-methyl tetrahydrofuran, methyl tert-butyl ether, di-tert-butyl ether, diisopropyl ether, diethyl ether, cyclohexane, 1,4-dioxane or a mixture thereof. In particular, the solvent is mixture of toluene and tetrahydrofuran.

In general, the reaction at step (c) may be performed in presence of an acid and a solvent or a mixture of solvents, wherein the acid is selected from hydrochloric acid, hydrobromic acid, sulfuric acid, nitric acid or mixture thereof. The solvent is selected from water, methanol, ethanol, isopropanol, n-butanol, acetone, methyl ethyl ketone, methyl isobutyl ketone, acetonitrile, dimethyl formamide, N,N-dimethyl acetamide, dimethyl sulfoxide, N-methyl pyrrolidone, ethyl acetate, isopropyl acetate, butyl acetate, toluene, xylene, ethyl benzene, methylene dichloride, chlorobenzene, tetrahydrofuran, 2-methyl tetrahydrofuran, methyl tert-butyl ether, di-tert-butyl ether, diisopropyl ether, diethyl ether, cyclohexane, 1,4-dioxane or mixture thereof. In particular, the solvent is isopropyl acetate.

In general, the condensation reaction at step (d) may be performed by using a coupling agent in presence of a base and a solvent or a mixture of solvents, wherein the coupling agent is selected from HOBt, HOAt, HATU, HBTU, TBTU, TPTU, TOTU, EDC, EDC·HCl, DCC, DIC, CDI, SOCl₂, oxalyl chloride, Vilsmeier reagent, phosphorous oxychloride or a mixture thereof. In particular, the coupling agent is EDC·HCl. The base is selected from imidazole, TEA, DIPEA, NMM, pyridine, NaOH, NaHCO₃, Na₂CO₃, K₂CO₃, K₃PO₄, N-methylpiperidine or a mixture thereof. In particular, the base is DIPEA. The solvent is selected from DMF, dimethylacetamide, ethyl acetate, ethanol, methanol, propanol, isopropyl alcohol, NMP, dichloromethane, toluene, THF, n-heptane, n-hexane, dioxane, di-n-butyl ether, acetonitrile, MTBE, DME, water or a mixture thereof. In particular, the solvent is DMF.

In another general aspect, there is provided a process for preparation of a compound of Formula (VII) comprising, converting a compound of Formula (VI) or a salt, an isomer or a mixture thereof to the compound of Formula (VII) or a salt, an isomer or a mixture thereof.

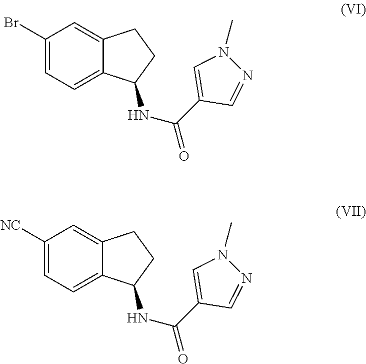 [SMILES: Cn1cc(C(=O)N[C@@H]2CCc3cc(Br)ccc32)cn1 ; Cn1cc(C(=O)N[C@@H]2CCc3cc(C#N)ccc32)cn1]

In general, the cyanation of the compound of Formula (VII) or a salt, an isomer or a mixture thereof to obtain the compound of Formula (VI) or a salt, an isomer or a mixture thereof, may be performed by using a reagent and a solvent or a mixture of solvents, wherein the reagent is selected from potassium ferrrocynide trihydrate, potassium ferrrocynide trihydrate with XPhos and potassium acetate, potassium ferrrocynide trihydrate with 2^{nd} Gen Xphos and potassium acetate, copper cyanide, sodium cyanide, potassium cyanide, dimethylmalononitrile, ethyl cyanoacetate, or mixture thereof. In particular, the reagent is potassium ferrrocynide trihydrate with XPhos and potassium acetate. The solvent is selected form water, methanol, ethanol, isopropanol, n-butanol, acetone, methyl ethyl ketone, methyl isobutyl ketone, acetonitrile, dimethyl formamide, N,N-dimethyl acetamide, dimethyl sulfoxide, N-methyl pyrrolidone, ethyl acetate, isopropyl acetate, butyl acetate, toluene, xylene, ethyl benzene, methylene dichloride, chlorobenzene, tetrahydrofuran, 2-methyl tetrahydrofuran, methyl tert-butyl ether, di-tert-butyl ether, diisopropyl ether, diethyl ether, cyclohexane, 1,4-dioxane, nitrobenzene, pyridine or a mixture thereof. In particular, the solvent is mixture of water and 1,4-dioxane.

In another general aspect, there is provided a process for preparation of compound of Formula (VII) comprising:

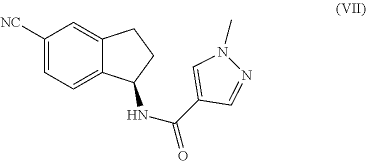 [SMILES: Cn1cc(C(=O)N[C@@H]2CCc3cc(C#N)ccc32)cn1]

(a) converting a compound of Formula (IV) or a salt, an isomer or a mixture thereof to a compound of Formula (VI) or a salt, an isomer or a mixture thereof, and

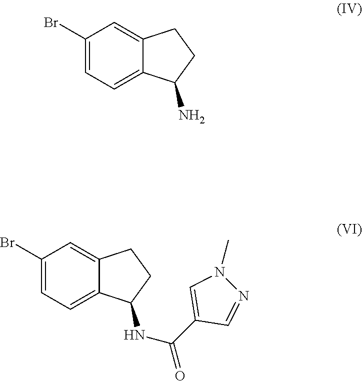 [SMILES: N[C@@H]1CCc2cc(Br)ccc21 ; Cn1cc(C(=O)N[C@@H]2CCc3cc(Br)ccc32)cn1]

(b) converting the compound of Formula (VI) or a salt, an isomer or a mixture thereof to the compound of Formula (VII) or a salt, an isomer or a mixture thereof.

In general, the step (a) involves condensation of compound of Formula (IV) or a salt, an isomer or a mixture thereof with a compound of Formula (V),

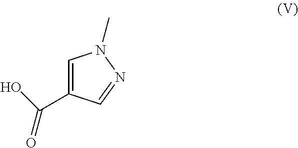

to obtain the compound of Formula (VI) or a salt, an isomer or a mixture thereof.

In general, the condensation of the compound of Formula (IV) or a salt, an isomer or a mixture thereof with the compound of Formula (V) may be performed in presence of a coupling agent, a base and a solvent or a mixture of solvents, wherein the coupling agent is selected from HOBt, HOAt, HATU, HBTU, TBTU, TPTU, TOTU, EDC, EDC·HCl, EDCI, DCC, DIC, CDI, SOCl₂, oxalyl chloride, Vilsmeier reagent, phosphorous oxychloride, or a mixture thereof. In particular, the coupling agent is EDC·HCl with HOBt. The base selected from imidazole, TEA, DIPEA, NMM, pyridine, NaOH, NaHCO₃, Na₂CO₃, K₂CO₃, K₃PO₄, N-methylpiperidine or a mixture thereof. In particular, the base is DIPEA. The solvent is selected from DMF, dimethylacetamide, ethyl acetate, ethanol, methanol, propanol, isopropyl alcohol, NMP, or dichloromethane, toluene, THF, n-heptane, n-hexane, dioxane, di-n-butyl ether, acetonitrile, MTBE, DME, water or a mixture thereof. In particular, the solvent is DMF.

In general, the cyanation reaction at step (b) may be performed by using a reagent and a solvent or a mixture of solvents, wherein the reagent is selected from potassium ferrrocynide trihydrate, potassium ferrrocynide trihydrate with XPhos and potassium acetate, potassium ferrrocynide trihydrate with 2^{nd} Gen Xphos and potassium acetate, copper cyanide, sodium cyanide, potassium cyanide, dimethylmalononitrile, ethyl cyanoacetate, or mixture thereof. In particular, the reagent is potassium ferrrocynide trihydrate with XPhos and potassium acetate. The solvent is selected form water, methanol, ethanol, isopropanol, n-butanol, acetone, methyl ethyl ketone, methyl isobutyl ketone, acetonitrile, dimethyl formamide, N,N-dimethyl acetamide, dimethyl sulfoxide, N-methyl pyrrolidone, ethyl acetate, isopropyl acetate, butyl acetate, toluene, xylene, ethyl benzene, methylene dichloride, chlorobenzene, tetrahydrofuran, 2-methyl tetrahydrofuran, methyl tert-butyl ether, di-tert-butyl ether, diisopropyl ether, diethyl ether, cyclohexane, 1,4-dioxane, nitrobenzene, pyridine or a mixture thereof. In particular, the solvent is mixture of water and 1,4-dioxane.

In another general aspect, there is provided a use of compound of Formula (VI) or a salt, an isomer or a mixture thereof for preparation of Aficamten.

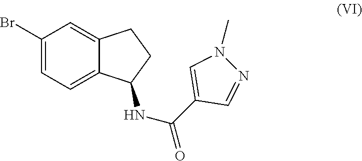 [SMILES: Cn1cc(C(=O)N[C@@H]2CCc3cc(Br)ccc32)cn1]

In another general aspect there is provided a process for preparation of Aficamten comprising:
(a) converting a compound of Formula (VI) or a salt, an isomer or a mixture thereof to compound of Formula (VII) or a salt, an isomer or a mixture thereof,

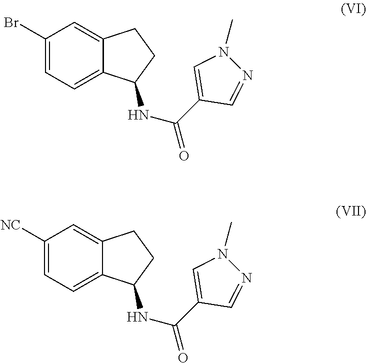 [SMILES: Cn1cc(C(=O)N[C@@H]2CCc3cc(Br)ccc32)cn1 ; Cn1cc(C(=O)N[C@@H]2CCc3cc(C#N)ccc32)cn1]

(b) converting the compound of Formula (VII) or a salt, an isomer or a mixture thereof to compound of Formula (VIII) or a salt, an isomer or a mixture thereof, and

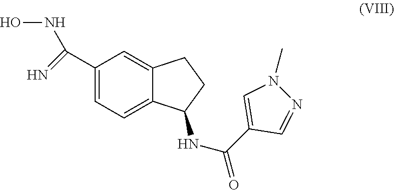 [SMILES: Cn1cc(C(=O)N[C@@H]2CCc3cc(C(=N)NO)ccc32)cn1]

(c) converting the compound of Formula (VIII) or a salt, an isomer or a mixture thereof to Aficamten.

In general, the cyanation reaction at step (a) may be performed by using a reagent and a solvent or a mixture of solvents, wherein the reagent is selected from potassium ferrrocynide trihydrate, potassium ferrrocynide trihydrate with XPhos and potassium acetate, potassium ferrrocynide trihydrate with 2^{nd} Gen Xphos and potassium acetate, copper cyanide, sodium cyanide, potassium cyanide, dimethylmalononitrile, ethyl cyanoacetate, or mixture thereof. In particular, the reagent is potassium ferrrocynide trihydrate with XPhos and potassium acetate. The solvent is selected form water, methanol, ethanol, isopropanol, n-butanol, acetone, methyl ethyl ketone, methyl isobutyl ketone, acetonitrile, dimethyl formamide, N,N-dimethyl acetamide, dimethyl sulfoxide, N-methyl pyrrolidone, ethyl acetate, isopropyl acetate, butyl acetate, toluene, xylene, ethyl benzene, methylene dichloride, chlorobenzene, tetrahydrofuran, 2-methyl tetrahydrofuran, methyl tert-butyl ether, di-tert-butyl ether, diisopropyl ether, diethyl ether, cyclohexane, 1,4-dioxane, nitrobenzene, pyridine or a mixture thereof. In particular, the solvent is mixture of water and 1,4-dioxane.

In general, the reaction at step (b) may be performed by using hydroxylamine, hydroxylamine hydrochloride in the presence of a base and a solvent or a mixture of solvents, wherein the base is selected from NaOH, NaHCO₃, Na₂CO₃, K₂CO₃, K₃PO₄, TEA, DIPEA, NMM, pyridine, N-methylpiperidine or a mixture thereof. In particular, the base is TEA. The solvent is selected from water, methanol, ethanol, isopropanol, n-butanol, acetone, methyl ethyl ketone, methyl isobutyl ketone, acetonitrile, dimethyl formamide, N,N-dimethyl acetamide, dimethyl sulfoxide, N-methyl pyrrolidone, ethyl acetate, isopropyl acetate, butyl acetate, toluene, xylene, ethyl benzene, methylene dichloride, chlorobenzene, tetrahydrofuran, 2-methyl tetrahydrofuran, methyl tert-butyl ether, di-tert-butyl ether, diisopropyl ether, diethyl ether, cyclohexane, 1,4-dioxane or a mixture thereof. In particular, the solvent is dimethyl formamide.

In general, cyclization reaction at step (c) may be performed by using propionic anhydride, propionic acid or mixture thereof, in presence of a solvent or a mixture of solvents, wherein the solvent is selected from water, methanol, ethanol, isopropanol, n-butanol, acetone, methyl ethyl ketone, methyl isobutyl ketone, acetonitrile, dimethyl formamide, N,N-dimethyl acetamide, dimethyl sulfoxide, N-methyl pyrrolidone, ethyl acetate, isopropyl acetate, butyl acetate, toluene, xylene, ethyl benzene, methylene dichloride, chlorobenzene, tetrahydrofuran, 2-methyl tetrahydrofuran, methyl tert-butyl ether, di-tert-butyl ether, diisopropyl ether, diethyl ether, cyclohexane, 1,4-dioxane, diglyme or a mixture thereof. In particular, the solvent is dimethyl formamide.

In another general aspect there is provided a process for preparation of Aficamten comprising:
(a) converting a compound of Formula (IV) or a salt, an isomer or a mixture thereof with a compound of Formula (V) to obtain a compound of Formula (VI) or a salt, an isomer or a mixture thereof,

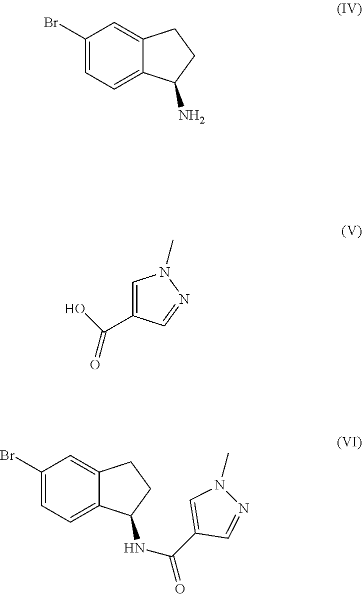

(b) converting the compound of Formula (VI) or a salt, an isomer or a mixture thereof to a compound of Formula (VII) or a salt, an isomer or a mixture thereof,

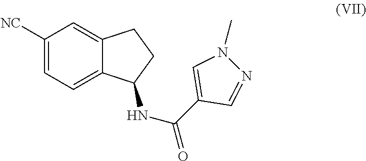 [SMILES: Cn1cc(C(=O)N[C@@H]2CCc3cc(C#N)ccc32)cn1]

(c) converting the compound of Formula (VII) or a salt, an isomer or a mixture thereof to a compound of Formula (VIII) or a salt, an isomer or a mixture thereof, and

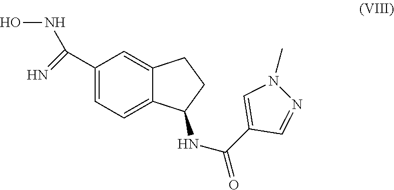 [SMILES: Cn1cc(C(=O)N[C@@H]2CCc3cc(C(=N)NO)ccc32)cn1]

(d) converting the compound of Formula (VIII) or a salt, an isomer or a mixture thereof to Aficamten.

In general, the condensation reaction at step (a) may be performed by using a coupling agent in presence of a base and a solvent or a mixture of solvents, wherein the coupling agent is selected from HOBt, HOAt, HATU, HBTU, TBTU, TPTU, TOTU, EDC, EDC·HCl, EDCI, DCC, DIC, CDI, SOCl₂, oxalyl chloride, Vilsmeier reagent, phosphorous oxychloride, acetic anhydride, Pivaloyl chloride, or a mixture thereof. In particular, the coupling agents is EDC·HCl with HOBt. The base is selected from imidazole, TEA, DIPEA, NMM, pyridine, NaOH, NaHCO₃, Na₂CO₃, K₂CO₃, K₃PO₄, N-methylpiperidine or a mixture thereof. In particular, the base is DIPEA. The solvent is selected from DMF, dimethylacetamide, ethyl acetate, ethanol, methanol, propanol, isopropyl alcohol, NMP, or dichloromethane, toluene, THF, n-heptane, n-hexane, dioxane, di-n-butyl ether, acetonitrile, MTBE, DME, water or a mixture thereof. In particular, the solvent is DMF.

In general, the cyanation reaction at step (b) may be performed by using a reagent and a solvent or mixture of solvents, wherein the reagent is selected from potassium ferrrocynide trihydrate, potassium ferrrocynide trihydrate with XPhos and potassium acetate, potassium ferrrocynide trihydrate with 2^{nd} Gen Xphos and potassium acetate, copper cyanide, sodium cyanide, potassium cyanide, dimethylmalononitrile, ethyl cyanoacetate, or mixture thereof. In particular, the reagent is potassium ferrrocynide trihydrate with XPhos and potassium acetate. The solvent is selected form water, methanol, ethanol, isopropanol, n-butanol, acetone, methyl ethyl ketone, methyl isobutyl ketone, acetonitrile, dimethyl formamide, N,N-dimethyl acetamide, dimethyl sulfoxide, N-methyl pyrrolidone, ethyl acetate, isopropyl acetate, butyl acetate, toluene, xylene, ethyl benzene, methylene dichloride, chlorobenzene, tetrahydrofuran, 2-methyl tetrahydrofuran, methyl tert-butyl ether, di-tert-butyl ether, diisopropyl ether, diethyl ether, cyclohexane, 1,4-dioxane, nitrobenzene, pyridine or a mixture thereof. In particular, the solvent is mixture of water and 1,4-dioxane.

In general, the reaction at step (c) may be performed by using hydroxylamine or hydroxylamine hydrochloride in presence of a base and a solvent or a mixture of solvents, wherein the base is selected from NaOH, NaHCO₃, Na₂CO₃, K₂CO₃, K₃PO₄, TEA, DIPEA, NMM, pyridine, N-methylpiperidine or a mixture thereof. In particular, the base is TEA. The solvent is selected from water, methanol, ethanol, isopropanol, n-butanol, acetone, methyl ethyl ketone, methyl isobutyl ketone, acetonitrile, dimethyl formamide, N,N-dimethyl acetamide, dimethyl sulfoxide, N-methyl pyrrolidone, ethyl acetate, isopropyl acetate, butyl acetate, toluene, xylene, ethyl benzene, methylene dichloride, chlorobenzene, tetrahydrofuran, 2-methyl tetrahydrofuran, methyl tert-butyl ether, di-tert-butyl ether, diisopropyl ether, diethyl ether, cyclohexane, 1,4-dioxane or a mixture thereof. In particular, the solvent is dimethyl formamide.

In general, cyclization reaction at step (d) may be performed by using propionic anhydride, propionic acid or mixture thereof, in presence of a solvent or a mixture of solvents, wherein the solvent is selected from water, methanol, ethanol, isopropanol, n-butanol, acetone, methyl ethyl ketone, methyl isobutyl ketone, acetonitrile, dimethyl formamide, N,N-dimethyl acetamide, dimethyl sulfoxide, N-methyl pyrrolidone, ethyl acetate, isopropyl acetate, butyl acetate, toluene, xylene, ethyl benzene, methylene dichloride, chlorobenzene, tetrahydrofuran, 2-methyl tetrahydrofuran, methyl tert-butyl ether, di-tert-butyl ether, diisopropyl ether, diethyl ether, cyclohexane, 1,4-dioxane, diglyme or mixture thereof. In particular, the solvent is dimethyl formamide.

In another general aspect there is provided a process for preparation of Aficamten comprising:
(a) converting a compound of Formula (I) to a compound of Formula (IV) or a salt, an isomer or a mixture thereof,

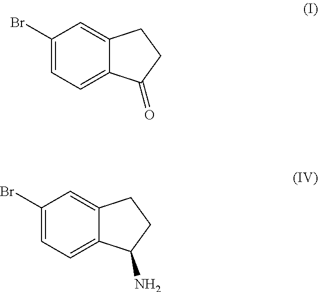 [SMILES: O=C1CCc2cc(Br)ccc21 ; N[C@@H]1CCc2cc(Br)ccc21]

(b) reacting the compound of Formula (IV) or a salt, an isomer or a mixture thereof with a compound of Formula (V) to obtain a compound of Formula (VI) or a salt, an isomer or a mixture thereof,

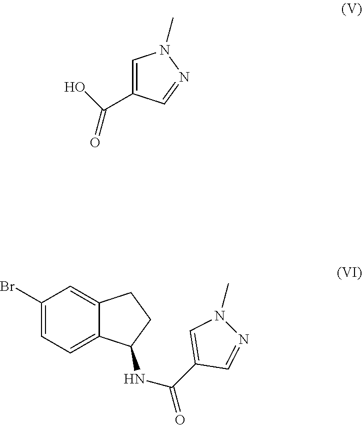

(c) converting the compound of Formula (VI) or a salt, an isomer or a mixture thereof to a compound of Formula (VII) or a salt, an isomer or a mixture thereof,

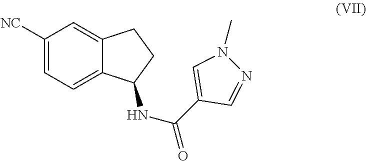 [SMILES: Cn1cc(C(=O)N[C@@H]2CCc3cc(C#N)ccc32)cn1]

(d) converting the compound of Formula (VII) or a salt, an isomer or a mixture thereof to a compound of Formula (VIII) or a salt, an isomer or a mixture thereof, and

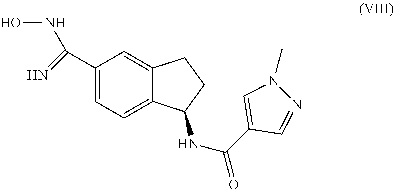 [SMILES: Cn1cc(C(=O)N[C@@H]2CCc3cc(C(=N)NO)ccc32)cn1]

(e) converting the compound of Formula (VIII) or a salt, an isomer or a mixture thereof to Aficamten.

In general, the conversion of the compound of Formula (I) to the compound of Formula (IV) or a salt, an isomer or a mixture thereof via formation of a compound of Formula (II) or a salt, an isomer or a mixture thereof and a compound of Formula (III) or a salt, an isomer or a mixture thereof.

The process of this aspect may be performed with isolation or without isolation of the compound of Formula (II) or a salt, an isomer or a mixture thereof.

The process of this aspect may be performed with isolation or without isolation of the compound of Formula (III) or a salt, an isomer or a mixture thereof.

In general, the conversion of compound of Formula (I) to compound of Formula (IV) or a salt, an isomer or a mixture thereof via formation of the compound of Formula (II-A) or a salt, an isomer or a mixture thereof and the compound of Formula (III-A) or a salt, an isomer or a mixture thereof.

The process of this aspect may be performed with isolation or without isolation of the compound of Formula (II-A) or a salt, an isomer or a mixture thereof.

The process of this aspect may be performed with isolation or without isolation of the compound of Formula (III-A) or a salt, an isomer or a mixture thereof.

The compounds of present invention can be in isolated and/or purified form, but such is not required.

The compounds of present invention include various physical forms of the salt including dissolved forms, oil or liquid forms, and solid forms.

The Aficamten may be isolated by precipitation, evaporation, spray drying, or other conventional techniques known in the art.

In another general aspect, the process of present invention provides Aficamten having a purity of about 98% or more by area percentage of HPLC. In particular, Aficamten having a purity of about 99% or more, more particularly, a purity of about 99.5% or more, further more particularly, a purity of about 99.8% or more, most particularly, a purity of about 99.9% or more, by area percentage of HPLC.

In another general aspect, the process of present invention provides Aficamten having a chiral purity of about 98% or more, by area percentage of HPLC. In particular, Aficamten having a chiral purity of about 99% or more, more particularly, a chiral purity of about 99.5% or more, furthermore, a chiral purity of about 99.8% or more, most particularly, a chiral purity of about 99.9% or more, by area percentage of HPLC.

In another general aspect, the present invention provides a composition comprising an Aficamten or pharmaceutically acceptable salt thereof and pharmaceutically acceptable carrier.

In another general aspect, there is provided a composition comprising Aficamten or pharmaceutically acceptable salt having a purity of about 99% or more, as determined by high performance liquid chromatography (HPLC).

In another general aspect, there is provided a composition comprising Aficamten or pharmaceutically acceptable salt having a chiral purity of about 99% or more, as determined by high performance liquid chromatography (HPLC).

In another general aspect, there is provided Aficamten having a purity of about 98% or more and a chiral purity of about 98% or more, as determined by high performance liquid chromatography (HPLC).

In another general aspect, there is the composition comprises Aficamten having a purity of 99% or more, and one or more of a compound of Formula (A), or a compound of Formula (B), or a compound of Formula (C), each present in an amount of about 0.15% or less, relative to Aficamten, by weight, when measured by area percentage of HPLC,

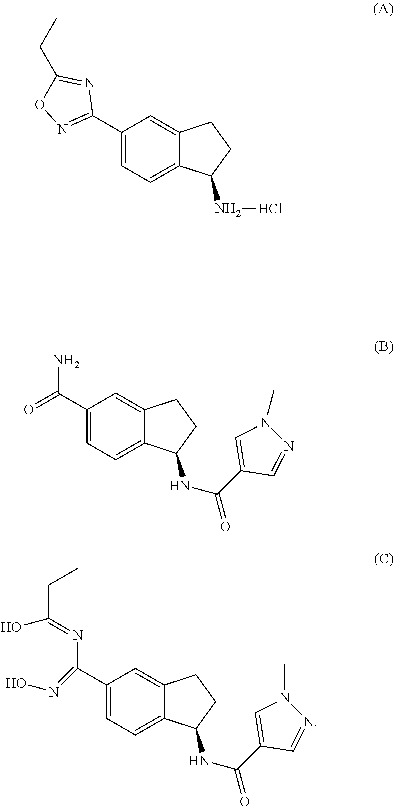 [SMILES: CCc1nc(-c2ccc3c(c2)CC[C@H]3NCl)no1 ; Cn1cc(C(=O)N[C@@H]2CCc3cc(C(N)=O)ccc32)cn1 ; CC/C(O)=N/C(=N\O)c1ccc2c(c1)CC[C@H]2NC(=O)c1cnn(C)c1]

TABLE 1
SUMMARY OF HPLC ANALYSIS OF AFICAMTEN

| | | Compound of | Compound of | Compound of |
|---|---|---|---|---|
| Sample | HPLC purity | Formula (A) | Formula (B) | Formula (C) |
| Aficamten | 99.94% | ND | ND | ND |

ND indicates “Not Detected”
Limit of Detection (LOD) = 0.02%

The purity of the samples of Aficamten is measured by area percentage of HPLC using following conditions:
Equipment: Shimadzu LC2020CHT HPLC system or equivalent
Column: Kromasil C18 (250 mm×4.6 mm, 5 μm)
Detector: UV-VIS detector or PDA detector
Flow rate: 1.0 mL/min
Wavelength: 205 nm
Injection Vol.: 10 μL
Column Oven Temp.: 40° C.
Run Time: 60 min

The present invention is further illustrated by following reaction examples which are provided merely to be representative of the invention and do not limit the scope of it.

### EXAMPLES

### Example 1: Process for Preparation of (R)-5-bromo-2,3-dihydro-1H-inden-1-amine (IV)

1.1: Process for preparation of (S)-5-bromo-2,3-dihydro-1H-inden-1-ol (II-A): In a 1-litre RBF, DCM (100 mL) and 5-Bromo indanone (10 g, 0.0473 mol) were added. Further, R—(+)-2-Methyl-CBS-oxazaborolidine (1M in Toluene) (7.1 mL) at −25 to −15° C. Borane dimethyl sulfide complex (10 M in THF) (5.7 mL) was then added and the resulting mass was stirred till TLC complies. Water (40 mL) was added and the reaction mass was stirred. Raise the temperature to 20-30° C. and the reaction mass was filtered through filter pad and the solid was washed with Dichloromethane (100 ml) at 20-30° C. The layers were separated and the organic layer was concentrated under vacuum followed by isolation from cyclohexane to obtain 8.5 g of the title compound (II-A).

1.2: Process for preparation of (R)-1-azido-5-bromo-2,3-dihydro-1H-indene (III-A): Compound (II-A) (4.2 g) and toluene (34 mL) were added into a 1-litre RBF at 25-35° C. the reaction mass was cooled to 0-10° C. and DPPA (6.51 g) was added followed by addition of DBU (4.5 g) under nitrogen within 1 hour. The resulting mass was stirred for additional 2-5 hours at 0-20° C. and then water (40 mL) was added. The resulting mass was stirred and the layers were separated. The organic layer was washed with water (2×40 mL), dried over anhydrous sodium sulfate, and concentrated under vacuum to obtain 4.44 g of the title compound (III-A).

1.3.1: process for preparation of (R)-5-bromo-2,3-dihydro-1H-inden-1-amine (IV): Compound (III-A) (4.4 g 0.186 mol, 100 eq.) and methanol (60 mL) were added into a 1-litre RBF. SnCl₂·2H₂O (5.0 g, 0.022 mol, 1.2 eq.) was then added into reaction mass and resulting mass was stirred till completion of the reaction. Raise temperature to 45-55° C. and distilled out methanol under vacuum. The vacuum was released and Ethyl acetate (20 mL) was charged and 2N NaOH solution (20 mL) were added into reaction mass and the mass was stirred at 25-35° C. for 1-2 hours. The salt was filtered and the filtrate was allowed to settle. The layers were separated. Aqueous phase was extracted by ethyl acetate (30 mL) and conc. HCl (2.5 g) was added into the combined organic layers at 0-10° C. The product was filtered and dried under vacuum to obtain 3.1 g of the title compound (IV).

1.3.2 process for preparation of (R)-5-bromo-2,3-dihydro-1H-inden-1-amine (IV): Compound (III-A) (28 g, 0.118 mol, 1.0 eq.) and THF (400 mL) were added into 1-litre RBF. PPh₃ (46.42 g, 0.177 mol, 1.5 eq.) was added into the reaction mass and stirred for 1 hour. The reaction mass was poured into a solution of potassium hydroxide (16.55 g, 0.295 mol, 2.5 eq.) in water (100 mL) and stirred for 3 hours. The temperature of the reaction mass was raised to 55° C. and stirred for 2 hours. The reaction mass was cooled to room temperature and ethyl acetate (500 mL) was added into it followed by addition of brine solution (200 mL). The aqueous layer was added to RBF and extracted with ethyl acetate (2×300 mL). The organic layers were combined and washed with brine solution (20%, 300 mL). The organic layer was dried over sodium sulfate and concentrated under vacuum to obtain 19.32 g (77%) of the title compound (IV).

1.3.3: process for preparation of (R)-5-bromo-2,3-dihydro-1H-inden-1-amine (IV): Compound (III-A) (28 g, 0.118 mol, 1.0 eq.) and 1,4-Dioxane (112 mL) were added into a RBF. The reaction mass was cooled to 10-20° C. and Zinc (30.86 g, 0.742 mol, 4.0 eq.) was added into it followed by addition of ammonium formate (37.2 g, 0.59 mol, 5 eq.) solution prepared in water (112 mL). The reaction mass was stirred for 2 hours. The reaction mass was cooled to 10-15° C. and liquor ammonia (56 mL) solution in water (240 mL) was added into it. DCM (200 mL) was added into reaction mass and filtered it through celite followed by washing with DCM (2×30 mL). Filtrate was separated and aqueous layer was extracted by DCM (2×100 mL). Organic layers were combined and washed with water (2×60 mL) followed by separation of the layers. The organic layer was dried over sodium sulfate and concentrated under vacuum to obtain 19.32 g (77%) of the title compound (IV).

### Example 2: Process for Preparation of (R)-5-bromo-2,3-dihydro-1H-inden-1-amine (iv) hydrochloride

2.1: Process for preparation of (R)—N—((R)-5-bromo-2,3-dihydro-1H-inden-1-yl)-2-methylpropane-2-sulfinamide (III): Toluene (16 mL), (R)-tert-Butane sulfinamide (5 g, 0.041 mol) and titanium ethoxide Ti(OEt)₄, (8.5 g, 0.04 mol) were added into a 1-litre RBF. The temperature of the mass was raised to 75-85° C. The solution of 5-Bromo-2,3-dihydro-1H-inden-1-one (3.9 g, 0.02 mol) in toluene (35 mL) was prepared and added into the reaction mass, while maintaining the temperature at 75-85° C. The reaction mass was stirred till completion of the reaction. The reaction mass was cooled to 20-30° C. and THF (40 mL) was added. Further, the reaction mass was cooled to −20 to −5° C. and portion-wise sodium borohydride (NaBH₄, 0.4 g, 0.01 mol) was added into it. The reaction mass was stirred at −15 to −5° C. for 10-15 hours. The temperature of reaction mass was raised to 25-35° C. and 2.5M solution of potassium glycolate (48.4 g) was added into it. The resulting reaction mass was stirred for 15-45 min and then layers were allowed to settle for 1-3 hours. The lower aqueous layer was separated and the solid retained with the organic layer. The organic layer was washed twice with 2.5M potassium glycolate (2×13 g) followed by 20% sodium chloride (2×15 g). The organic layer was filtered through Hyflo bed and washed the bed with toluene (2×5 ml). The organic layers were combined, concentrated under vacuum and stripped out with ethyl acetate (EtOAc, 15 ml). Ethyl acetate (15 mL) was added into the concentrate mass charge at 60-70° C. and the solution was stirred at the same temperature for 1-2 hours. n-heptane (25 mL) was added into the resulting mass and maintained for 1-2 hours. The reaction mass was cooled to 0-10° C. for 2-4 hours. The solid was filtered and washed the wet cake with prechilled mass of EtOAc (3 mL) and n-heptane (8 mL). The product was dried at 30-50° C. for 8-10 hours to obtain 4.2 g of the tile compound (III).

2.2: Process for preparation of (R)-5-bromo-2,3-dihydro-1H-inden-1-amine (IV) hydrochloride: Isopropyl acetate (IPAc) (110 mL) and compound (III) (4.2 g, 0.013 mol) were treated with 6M HCl (1.8 g, 0.016 mol) in a 1-litre RBF. The resulting mass was stirred for 15-20 hours. After completion of the reaction, the product was filtered. The wet cake was washed with IPAc (15 mL) and dried at 35-50° C.

### Example 3: Process for Preparation of (R)—N-(5-bromo-2,3-dihydro-1H-inden-1-yl)-1-methyl-1H-pyrazole-4-carboxamide (VI)

DMF (300 mL), 1-methyl-1H-pyrazole-4-carboxylie acid (6.1 g, 48.4 mmol) and DIPEA (12.6 g, 121 mmol) were added into a 1-litre RBF. HOAt (19.75 g, 145.1 mmol) and EDC-HCl (28 g, 145.1 mmol) were then added into the reaction mass followed by addition of compound (IV) (12 g, 48.4 mmol). The reaction mass was stirred for 1-5 hours. After completion of the reaction, DCM was added into the reaction mass and washed it with NH₄Cl solution three times followed by washing with water. The organic layer was concentrated under reduced pressure and the product was isolated by mass of Ethyl acetate:Cyclohexane. The obtained crude product was further triturated with mass of Ethyl acetate:Cyclohexane (1:10) to obtain 12 g (87%) of title compound (VI).

### Example 4: Process for Preparation of (R)—N-(5-bromo-2,3-dihydro-1H-inden-1-yl)-1-methyl-1H-pyrazole-4-carboxamide (VI)

DMF (50 mL) and 1-methyl-1H-pyrazole-4-carboxylie acid (5.92 g, 0.04 mol) were added into a 1-litre RBF. DIPEA (15.64 g, 0.12 mol), HOBt (8.16 g, 0.06 mol) and EDC·HCl (19.3 g, 0.1 mol) were then added into the reaction mass. Compound (IV) (10 g, 0.04 mol) was added into the reaction mass and stirred it for 1-6 hours or till the process comply (compound (IV) not more than 0.50% w/w by HPLC). After completion of the reaction, cool the reaction mass to 15-25° C. and added HCl solution [Conc. HCl (20 ml) in water (80 ml)] and water (150 ml). Filter the product and wet product was slurred with sodium bicarbonate solution sodium bicarbonate (3.4 g) in water (60 mL). The product was filtered and dried under vacuum to obtain 12 g title compound (VI).

### Example 5: Process for Preparation of (R)—N-(5-bromo-2,3-dihydro-1H-inden-1-yl)-1-methyl-1H-pyrazole-4-carboxamide (VI)

Solution of 1-methyl-1H-pyrazole-4-carboxylie acid (6.1 g, 48.4 mmol) in DCM (300 ml), catalytic amount of DMF and Thionyl chloride (6.9 g, 58.03 mmol) were added into a 1-lite RBF. The reaction mass was stirred till the acid compound observed almost nil. The reaction mass was concentrated under vacuum and diluted with DCM (200 ml). Compound (IV) (12 g, 48.4 mmol) in DCM (200 ml) and TEA (12.24 g, 121 mmol) was added into another RBF and the reaction mass was cooled to 0-15° C. To the cooled reaction mass, above prepared acid chloride solution in DCM was added at 0-15° C. and stirred it for 3 hours. Water was added and reaction mass was stirred for 10-20 min. followed by separation of layers. The organic layer was washed again with water and the organic layer was concentrated under reduced pressure followed by isolation of the product by mass of ethyl acetate:cyclohexane. The obtained crude product was further triturated with mass of ethyl acetate:cyclohexane (1:10) to obtain 10-12 g (87%) of title compound (VI).

### Example 6: Process for Preparation of (R)—N-(5-cyano-2,3-dihydro-1H-inden-1-yl)-1-methyl-1H-pyrazole-4-carboxamide (VII)

DMF (10 mL), compound (VI) (1.4 g, 4.4 mmol), and CuCN (587 mg, 6.6 mmol) were added into the 1-litre RBF. The temperature of the reaction mass was raised to 150-160° C. and stirred for 2 days. The progress of the reaction was monitored by TLC. After completion of reaction, EtOAc (250 mL) was added and washed the reaction mass with water followed by 20% brine solution (3×150 mL). The layers were separated and organic layer was dried with sodium sulfate. The organic layer was concentrated under reduced pressure, and compound (VII) was isolated from mass of ethyl acetate:cyclohexane to obtain 0.55 g of compound (VII).

### Example 7: Process for Preparation of (R)—N-(5-cyano-2,3-dihydro-1H-inden-1-yl)-1-methyl-1H-pyrazole-4-carboxamide (VII)

1,4-Dioxane (200 mL) and compound (VI) (25 g, 78.07 mmol) were added into RBF. K₄Fe(CN)₆3H₂O (16.5 g, 39.05 mmol), 2nd Generation XPhos precatalyst (1.23 g, 0.16 mmol,), X-phos (0.37 g, 0.08 mmol) and a solution of KOAc (15.32 g, 15.62 mmol) in water (200 mL) were then added under nitrogen. The temperature of the reaction mass was raised to 80-90° C. and the reaction mass was stirred for 1-3 hours or till the process comply (compound VI not more than 0.15% w/w by HPLC). After completion of the reaction, activated charcoal (1 to 10% w/w of compound (VI)) and Ethyl acetate (200 ml) was added into the reaction mass and hot filtered through hyflo bed. to remove the salt and activated charcoal. The filtrate was allowed to settle and layers were separated. Aqueous layer was extracted by ethyl acetate (2×75 mL). The combine organic layer was washed with brine (75 mL), and concentrated under reduced pressure. The residue was purified by mass of ethyl acetate (100 mL) to obtain 18.8 g of title compound (VII).

### Example 8: Process for Preparation of (R)—N-(5-(N-hydroxycarbamimidoyl)-2,3-dihydro-1H-inden-1-yl)-1-methyl-1H-pyrazole-4-carboxamide (VIII)

Ethanol (25 mL), compound (VII) (3.0 g, 11.3 mmol) and hydroxylamine (50% w/w in water, 4.0 mL) were added into a 1-litre RBF at 25-35° C. Temperature of the reaction mass was raised to 80° C. and the reaction mass was stirred till completion of the reaction. The reaction mass was concentrated to obtain title compound (VIII) (3.3 g) (98%), as an off-white solid.

### Example 9: Process for Preparation of (R)—N-(5-(N-hydroxycarbamimidoyl)-2,3-dihydro-1H-inden-1-yl)-1-methyl-1H-pyrazole-4-carboxamide (VIII)

N,N-dimethyl formamide (175 mL) and compound (VII) (25 g, 9.4 mol) were added in to RBF followed by addition of hydroxyl amine hydrochloride (19.57 g, 28.16 mol). TEA (29.45 g, 29.1 mol) was added at 25-35° C. into reaction mass. Temperature of the reaction mass was raised to 38-42° C. and stirred for 15-18 hours or till the process comply (compound VII not more than 0.50% w/w by HPLC). Add methanol (75 mL) and cool the reaction mass to 0-10° C. Filter the product and wet solid was slurred with water (250 mL), filter and dried under vacuum to obtain 23 g of title compound (VIII).

### Example 10: Process for Preparation of Aficamten

N,N-dimethylformamide (175 ml) and compound (VIII) (25 g, 0.08352 mol) were added into a 1-litre RBF. Propionic anhydride (12.5 g, 0.096 mol) followed by triethyl amine (10.56 g, 0.1 mol) was then added into reaction mass at 25-35° C. The temperature of reaction mass was raised to 80-90° C. and stirred for 15-18 hours or till the process comply (compound (VIII) not more than 0.50% w/w by HPLC). Cooled to 25-35° C. and water (525 ml) was added into it. The product was filtered and the wet product was dried under vacuum to obtain Aficamten (27.5 g), as a white solid having a purity of 99.94% by HPLC and a chiral purity of 100% by HPLC.

While the present invention has been described in terms of its specific embodiments, certain modification and equivalents will be apparent to those skilled in art and the intended to be included within the scope of the invention.

We claim:

1. A compound of Formula (VI) or a salt, an isomer, or a mixture thereof,

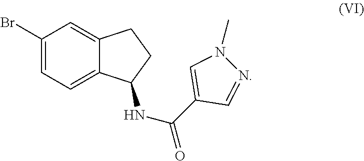 [SMILES: Cn1cc(C(=O)N[C@@H]2CCc3cc(Br)ccc32)cn1]

2. A process for the preparation of a compound of Formula (VI) or a salt, an isomer, or a mixture thereof, 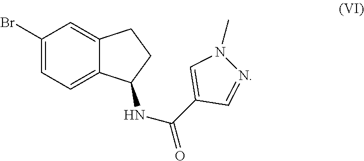 [SMILES: Cn1cc(C(=O)N[C@@H]2CCc3cc(Br)ccc32)cn1]
the process comprising reacting a compound of Formula (IV) or a salt, an isomer, or a mixture thereof, 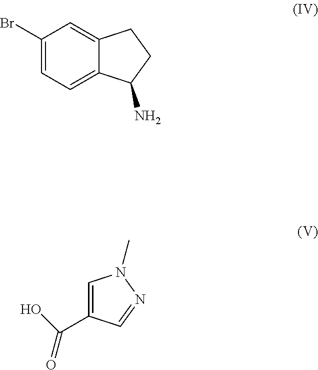
with a compound of Formula (V) to obtain the compound Formula (VI) or a salt, an isomer, or a mixture thereof.

3. The process according to claim 2, wherein the reaction is performed by using a coupling agent in the presence of a base and a solvent or a mixture of solvents, wherein the coupling agent is selected from EDC·HCl, HOBt, HOAt, HATU, thionyl chloride, CDI, DIC, DCC, sulfuryl chloride, phosphorous oxychloride, oxalyl chloride or a mixture thereof; the base is selected from TEA, imidazole, DIPEA, NMM, pyridine or a mixture thereof, and the solvent is selected from dimethylformamide, dimethylacetamide, ethyl acetate, ethanol, methanol, propanol, isopropyl alcohol, NMP, dichloromethane, toluene, tetrahydrofuran, n-heptane, n-hexane, dioxane, di-n-butyl ether, acetonitrile, MTBE, DME, water, or a mixture thereof.

4. A process for the preparation of Aficamten, the process comprising:
(a) converting a compound of Formula (VI) or a salt, an isomer, or a mixture thereof to a compound of Formula (VII) or a salt, an isomer, or a mixture thereof, 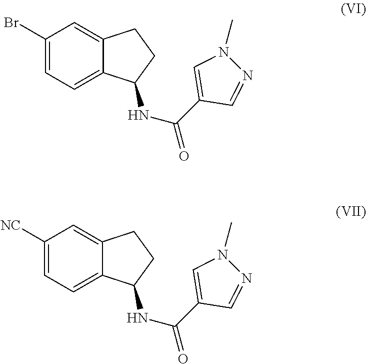 [SMILES: Cn1cc(C(=O)N[C@@H]2CCc3cc(Br)ccc32)cn1 ; Cn1cc(C(=O)N[C@@H]2CCc3cc(C#N)ccc32)cn1]
(b) converting the compound of Formula (VII) or a salt, an isomer, or a mixture thereof to a compound of Formula (VIII) or a salt, an isomer, or a mixture thereof; and 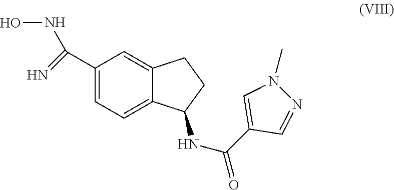 [SMILES: Cn1cc(C(=O)N[C@@H]2CCc3cc(C(=N)NO)ccc32)cn1]
(c) converting the compound of Formula (VIII) or salt, an isomer, or a mixture thereof to Aficamten

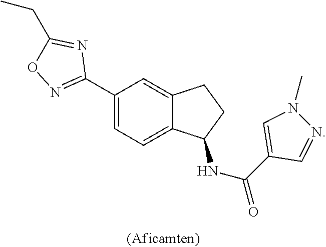 [SMILES: CCc1nc(-c2ccc3c(c2)CC[C@H]3NC(=O)c2cnn(C)c2)no1]

5. The process according to claim 4, wherein the reaction at step (a) is performed in the presence of a reagent in one or more solvent or a mixture of solvents, wherein the reagent comprises one or more of copper cyanide, sodium cyanide, potassium ferrocyanide trihydrate, potassium ferrocyanide trihydrate with XPhos and potassium acetate, potassium ferrocyanide trihydrate with 2nd Gen Xphos and potassium acetate, potassium cyanide, dimethylmalononitrile, ethyl cyanoacetate, or a mixture thereof, and the solvent comprises one or more of water, 1,4-dioxane, methanol, ethanol, isopropanol, n-butanol, acetone, methyl ethyl ketone, methyl isobutyl ketone, acetonitrile, dimethyl formamide or a mixture thereof

6. The process according to claim 4, wherein the reaction at step (b) is performed by using hydroxylamine, hydroxylamine hydrochloride or hydroxyl amine hydrosulphate in the presence of a base and a solvent or a mixture of solvents, wherein the base is selected from NaOH, NaHCO₃, Na₂CO₃, K₂CO₃, TEA, DIPEA, DIPEA or a mixture thereof, and the solvent comprises one or more of water, methanol, ethanol, isopropanol, n-butanol, acetone, methyl ethyl ketone, DMF, tetrahydrofuran, or a mixture thereof.

7. The process according to claim 4, wherein the reaction at step (c) is performed by using propionic anhydride, propionic acid or a mixture thereof, in the presence of a solvent or a mixture of solvents selected from one or more of water, DMF, methanol, ethanol, isopropanol, n-butanol, acetone, 1,4-dioxane, acetonitrile, or a mixture thereof.

8. A process for the preparation of Aficamten, the process comprising:
(a) converting a compound of Formula (I) to a compound of Formula (IV) or a salt, an isomer, or a mixture thereof; 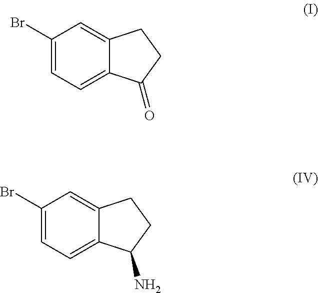 [SMILES: O=C1CCc2cc(Br)ccc21 ; N[C@@H]1CCc2cc(Br)ccc21]
(b) reacting the compound of Formula (IV) or a salt, an isomer, or a mixture thereof, with a compound of Formula (V) to obtain a compound Formula (VI) or a salt, an isomer, or a mixture thereof; and 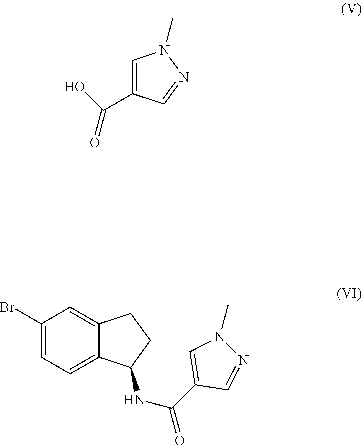
(c) converting the compound of Formula (VI) or a salt, an isomer or a mixture thereof to Aficamten

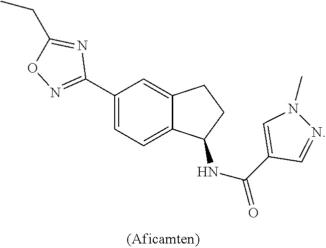 [SMILES: CCc1nc(-c2ccc3c(c2)CC[C@H]3NC(=O)c2cnn(C)c2)no1]

9. The process according to claim 8, wherein the conversion of the compound of Formula (I) to the compound Formula (IV) at step (a) is performed via the formation of compound of Formula (II), and a compound of Formula (III), 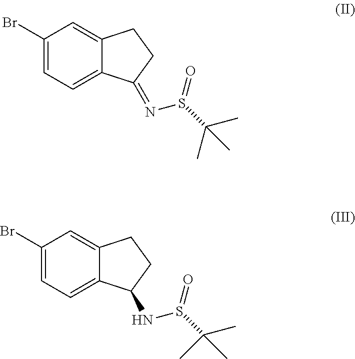
wherein, the process is performed with or without isolation of compound of Formula (II) and/or compound of Formula (III),
or alternatively, the conversion of the compound of Formula (I) to the compound of Formula (IV) is performed via the formation of a compound of Formula (II-A), 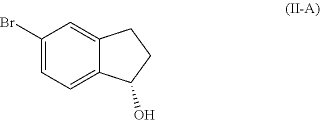 [SMILES: O[C@H]1CCc2cc(Br)ccc21]
and a compound of Formula (III-A), 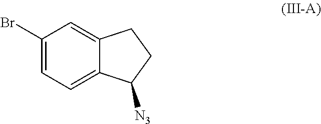
wherein, the process is performed with or without isolation of compound of Formula (II-A) and/or compound of Formula (III-A).

10. The process according to claim 8, wherein the reaction at step (b) is performed in the presence of a coupling agent and a base in one or more solvent or a mixture of solvents, wherein the coupling agent is selected from EDC·HCl, HOBt, HOAt, HATU, thionyl chloride, CDI, DCC, DCI, sulfuryl chloride, phosphorous oxychloride, oxalyl chloride or a mixture thereof; the base is selected from TEA, imidazole, DIPEA, NMM, pyridine or a mixture thereof; and the solvent is selected from DMF, dimethylacetamide, ethyl acetate, THF, or a mixture thereof.

11. A method of using a compound of Formula (VI) or a salt, an isomer, or a mixture thereof, 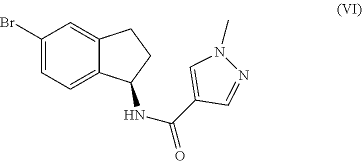 [SMILES: Cn1cc(C(=O)N[C@@H]2CCc3cc(Br)ccc32)cn1]
for the preparation of Aficamten, the method comprising converting the compound of Formula (VI) or a salt, an isomer or a mixture thereof to Aficamten

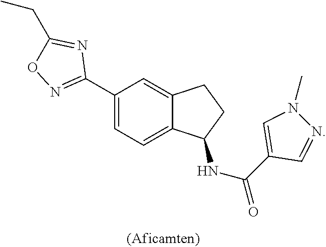 [SMILES: CCc1nc(-c2ccc3c(c2)CC[C@H]3NC(=O)c2cnn(C)c2)no1]

12. Aficamten having a purity of about 98% or more and a chiral purity of about 98% or more as determined by high performance liquid chromatography (HPLC).

13. A composition comprising the Aficamten of claim 11, the Aficamten having a purity of about 98% or more and a chiral purity of about 98% or more as determined by high performance liquid chromatography (HPLC).

14. The composition according to claim 13, wherein the Aficamten is having a purity of 99% or more, and one or more of a compound of Formula (A), or a compound of Formula (B), or a compound of Formula (C), each present in an amount of about 0.15% or less relative to Aficamten, by weight, when measured by area percentage of HPLC, 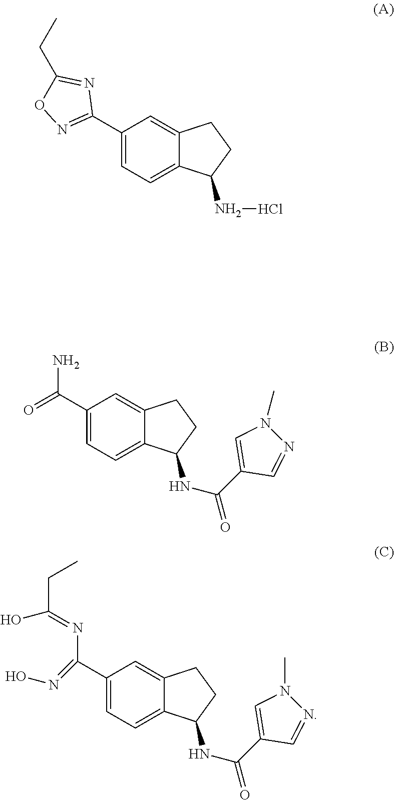 [SMILES: CCc1nc(-c2ccc3c(c2)CC[C@H]3NCl)no1 ; Cn1cc(C(=O)N[C@@H]2CCc3cc(C(N)=O)ccc32)cn1 ; CC/C(O)=N/C(=N\O)c1ccc2c(c1)CC[C@H]2NC(=O)c1cnn(C)c1]
